(19) United States
(12) Patent Application Publication
(10) Pub. No.: US 2021/0154368 A1
(43) Pub. Date: May 27, 2021

(54) BIOINK AND CROSSLINKABLE SUPPORT MEDIUM FOR PRINTING

(71) Applicant: CASE WESTERN RESERVE UNIVERSITY, Cleveland, OH (US)

(72) Inventors: Eben Alsberg, Cleveland, OH (US); Oju Jeon, Cleveland, OH (US); Yu Bin Lee, Cleveland, OH (US); Hyeon Jeong, Cleveland, OH (US)

(21) Appl. No.: 17/046,129

(22) PCT Filed: Apr. 9, 2019
(86) PCT No.: PCT/US19/26678

§ 371 Date: Oct. 8, 2020

Related U.S. Application Data
(60) Provisional application No. 62/655,136, filed on Apr. 9, 2018.
(60) Provisional application No. 62/744,900, filed on Oct. 12, 2018.

(51) Int. Cl. A61L 27/36; C08L 5/04; C09D 105/04; A61L 27/38; A61L 27/20; A61L 27/52; C12N 5/077; C12N 5/00; B33Y 70/00; B33Y 10/00

(52) U.S. Cl. CPC A61L 27/3691; C08L 5/04; C09D 105/04; A61L 27/3834; A61L 27/20; B33Y 80/00; C12N 5/0669; C12N 5/0062; B33Y 70/00; B33Y 10/00; C12N 2513/00; A61L 27/52

(57) ABSTRACT

A system for forming a scaffold-free 3D tissue construct includes a three dimensional (3D) printer; a self-healing, shear thinning, crosslinkable, biocompatible hydrogel support medium; and a first bioink that includes a plurality of cells. The first bioink is capable of being printed with the 3D printer into the hydrogel support medium in a defined shape.

### RELATED APPLICATION

This application claims priority from U.S. Provisional Application No. 62/655,136, file Apr. 9, 2018 and U.S. Provisional Application No. 62/744,900, filed Oct. 12, 2018, the subject matter of which are incorporated herein by reference in their entirety.

### BACKGROUND

Over the past decades, scaffolding approaches have been widely used to create functional tissues or organs in tissue engineering and regenerative medicine fields. However, the use of biomaterial-based scaffolds faces several challenges, such as interference with cell-cell interactions, potential immunogenicity of the materials and their degradation byproducts, unsynchronized rates of scaffold degradation with that of new tissue formation, and inhomogeneity and low density of seeded cells. To overcome these limitations of scaffold-based approaches, scaffold-free tissue engineering has recently emerged as a powerful strategy for constructing tissues using multicellular building blocks that self-assemble into geometries such as aggregates, sheets, strands and rings. These building blocks have been organized and fused into larger and more complicated structures, sometimes comprised of multiple cell types, and then they produce extracellular matrix (ECM) to form mechanically functional three-dimensional (3D) tissue constructs. However, it is still difficult to precisely control the architecture and organization of cell-only condensations to mimic sophisticated 3D structures of natural tissues and their structure-derived functions.

Recently, 3D printing has been applied in tissue engineering with the potential to create complicated 3D structures with high resolution using cell-free or cell-laden bioinks. Digital imaging data, obtained from computed tomography scans and magnetic resonance imaging, provide instruction for the desired geometry of printed constructs. Biodegradable thermoplastics, such as polycaprolactone, polylactic acid, and poly(lactic-co-glycolic acid), are advantageous for printing as stable constructs with delicate structural control can be formed due to the mechanical integrity of original materials. However, a major drawback is that cells cannot be printed simultaneously due to the use of organic solvents or high temperature to extrude the polymer inks.

### SUMMARY

Embodiments described herein relate to systems and methods for three dimensional (3D) bioprinting living cells to form scaffold-free 3D cell or tissue constructs. The 3D tissue constructs can be used in regenerative medicine, cell-based technologies, tissue engineering, and bioprinting applications. Unlike previous 3D bioprinting techniques which depend on external solid materials for structural maintenance or additional process for prefabrication of cell aggregates, the systems and methods described herein use a self-healing, sheer thinning, crosslinkable, biocompatible hydrogel support medium to provide a structural support role for the printed cell constructs, allowing media provision, and long-term culture. Precise maintenance of the structure, for example, mirroring an original computer aided design (CAD) file, can also be achieved even after maturation of the tissue by cell proliferation, differentiation, and extracellular matrix (ECM) production. The self-healing, sheer thinning, crosslinkable, biocompatible hydrogel support medium can be readily removed or separated from the 3D tissue construct by simple agitation or spontaneous degradation, and the cultured 3D constructs can be readily harvested from the self-healing, sheer thinning, crosslinkable, biocompatible hydrogel support medium without damage. The 3D bioprinting system and methods described herein make it possible to print isolated cells without a biomaterial carrier in the bioink, and can contribute to regenerative medicine by permitting generation of biomimetic cellular condensation-based engineered tissues with defined geometries comprised of multiple cell types with controlled spatial placement.

In some embodiments, a system for forming a scaffold-free 3D tissue construct includes a 3D printer, a self-healing, shear thinning, crosslinkable, biocompatible hydrogel support medium, and a first bioink. The first bioink includes a plurality of cells and optional macromer carrier, nanoparticles, microparticles, bioactive agents, cell aggregates, and/or organoids. The first bioink is capable of being printed with the 3D printer into the hydrogel support medium. The hydrogel support medium is capable of maintaining the printed first bioink in a defined shape during printing and optionally during culturing of the plurality of cells.

In some embodiments, the system can further include an additional bioink, such as a second, third, fourth, or more bioink, that is different than the first bioink. The additional bioink can include a plurality of cells, a macromer solution, nanoparticles, microparticles, bioactive agents, cell aggregates, organoids and/or combinations thereof. The additional bioink can be capable of being printed with the 3D printer into the hydrogel support medium.

The hydrogel support medium can behave as a viscous fluid during printing and be resistant to flow before and after printing. For example, initially, the hydrogel support medium is in a flow-resistant or solid-like state before being printed with the bioink. The hydrogel support medium becomes fluidized under the increased shear stress caused by printing the bioink into the hydrogel support medium. Then, after the printing is finished and the increased shear stress is removed, the hydrogel support medium can self-heal and form a flow-resistant or solid-like stable support medium.

In some embodiments, the hydrogel can be cytocompatible and, upon degradation, produces substantially non-toxic products.

In other embodiments, the system can further include a culture medium in which the hydrogel support medium can be cultured after the bioink is printed and the hydrogel support medium is further crosslinked. The culture medium can include bioactive agents for promoting growth and/or differentiation of the cells of the printed bioink. For example, the culture medium can include a cell differentiation medium.

In some embodiments, the hydrogel support medium can include a plurality of hydrogel particles that include a plurality of crosslinkable biodegradable natural polymer macromers. The hydrogel particles can have an average diameter of about 10 nm to about 10 mm. The natural polymer macromers can be at least partially crosslinked.

In some embodiments, the natural polymer macromers can include a plurality of acrylated and/or methacrylated natural polymer macromers. The acrylated and/or methacrylated, natural polymer macromers can be polysaccharides, which are optionally oxidized to aldehyde saccharide units, such as oxidized, acrylated and/or methacrylated alginates.

In other embodiments, the natural polymer macromers can be ionically crosslinked and can then be later photocrosslinked to enhance the mechanical stability of the hydrogel support medium.

In some embodiments, the plurality of cells can include progenitor cells, undifferentiated cells, differentiated cells, and/or cancer cells.

In other embodiments, the first bioink can be free of or substantially free of the optional macromer carrier, nanoparticles, microparticles, bioactive agents, cell aggregates, and/or organoids. The first bioink can be in a liquid or slurry form during printing.

Other embodiments relate to a method for forming a scaffold-free 3D tissue construct. The method can include providing a self-healing, shear thinning, crosslinkable, biocompatible hydrogel support medium. A first bioink can be printed into the hydrogel support medium in a defined shape. The first bioink can include a plurality of cells and optional macromer carrier, nanoparticles, microparticles, bioactive agents, cell aggregates, and/or organoids. The hydrogel support medium can be further crosslinked after printing to maintain the defined shape of the printed first bioink in the hydrogel support medium. The printed bioink, which includes a plurality of cells and optional macromer carrier, nanoparticles, microparticles, bioactive agents, cell aggregates, and/or organoids, can be cultured in the hydrogel support medium to form a cell aggregate or tissue construct structure with the defined shape.

In some embodiments, the self-healing, sheer thinning, crosslinkable, biocompatible hydrogel support medium can be readily removed or separated from the cell aggregate or tissue construct by, for example, simple agitation or spontaneous degradation, and the cell aggregate or tissue constructs can be readily harvested from the self-healing, sheer thinning, crosslinkable, biocompatible hydrogel support medium without damage.

In other embodiments, the method can include printing an additional bioink, such as a second, third, fourth or more bioink, into the hydrogel support medium. The additional bioink can be different than the first bioink and include a plurality of cells, cell aggregates, a macromer carrier, nanoparticles, microparticles, bioactive agents, organoids, and/or combinations thereof.

### BRIEF DESCRIPTION OF THE DRAWINGS

FIG. 1 is a schematic illustration of a 3D bioink printing system in accordance with an embodiment.

FIGS. 2(A-F) illustrate shear-thinning and self-healing alginate microgel supporting medium for 3D bioprinting of living stem cells. (A) A schematic of 3D printing of cells within the alginate microgel supporting medium. OMA microgels in the supporting medium fluidize via their shear-thinning properties when stress is applied by motion of the printing needle and cell-only bioink (shear-thinning region) and rapidly fill in after the needle passes by self-healing properties (self-healing region) without creating crevasses. Microgel supporting medium without shear stress presents solid-like properties, which provide mechanical stability for the printed cell construct (stable region). (B) Captured images at different times during bioprinting of the letter “C” using living stem cell-only bioink into the alginate microgel supporting medium. As the printing progress, cells are arranged into the letter “C” shape in 3D without disturbing previously printed regions, which is achieved as a result of the shear-thinning and self-healing properties of the alginate microgel supporting medium. (C-F) Images of the 3D bioprinted structures of a letter “C”, a cube, letters comprising the acronym “CWRU”, and a femur in alginate microgel supporting medium. Scale bars indicate 5 mm.

FIGS. 3(A-H) illustrate plots showing shear-thinning and self-healing properties of the alginate microgel supporting medium. (A) Storage (G′) and loss (G″) moduli of alginate microgel supporting medium (mean microgel diameter=7.0±2.8 μm) as a function of frequency. G′ is larger than G″ over the measured frequency range and both moduli exhibit frequency independence. Viscosity measurements of alginate microgel supporting medium as a function of (B) shear rate and (C) shear strain demonstrate its shear-thinning behavior. (D) G′ and G″ of the alginate microgel supporting medium as a function of shear strain exhibit its shear-yielding behavior and gel-to-sol transition at higher shear strain. (E) Shear moduli and (F) viscosity changes in dynamic strain tests of the alginate microgel supporting medium with alternating low (1%) and high (100%) strains at 1 Hz demonstrate its rapid recovery of strength and viscosity within seconds, which indicates “self-healing” or thixotropic properties. (G) Frequency sweep (at 1% strain) and (H) strain sweep (at 1 Hz) tests of the alginate microgel supporting medium after photocrosslinking under low-level UV light. G′ is larger than G″ over the measured frequency and strain ranges and both moduli exhibit frequency and strain independence, indicating that the photocrosslinked alginate microgel supporting medium is mechanically stable.

FIGS. 4(A-R) illustrate images and graphs showing the characterization of living cell bioink. (A-C) Live/Dead staining of 3D hMSC filaments bioprinted in a straight line, a corner and a curve with a 22 G needle and (D) their diameter distribution in the smaller alginate microgel supporting medium. (E-G) Live/Dead staining of 3D hMSC filaments bioprinted in various configurations with a 22 G needle and (H) their diameter distribution in the larger alginate microgel supporting medium. Arrows indicate the direction of movement of the printing nozzle. Scale bars indicate 600 μm. The Live/Dead images demonstrate high cell viability. Smaller alginate microgels lead to higher resolution printing by limiting diffusion of cells into the pores of the microgel bath. Thickness of the cell filaments also are more narrowly distributed in smaller microgel medium. Images of letters ‘C’ and “CWRU” in (I and J) the smaller and (K and L) larger alginate microgel supporting medium after photocrosslinking. Scale bars indicate 5 mm. (M-O) Live/Dead staining of 3D hMSC filaments bioprinted in various configurations with a 25 G needle and (P) their diameter distribution in the smaller alginate microgel supporting medium. (Q-R) Live/Dead staining of 3D hMSC filaments bioprinted in various configurations with a 27 G needle and (h) their diameter distribution in the smaller alginate microgel supporting medium. Scale bars indicate 600 μm. Smaller diameter needles lead to higher resolution printing of the cell filaments, which also are more narrowly distributed. Scale bars indicate 600 μm.

FIGS. 5(A-H) illustrate differentiation of 3D bioprinted hMSC constructs. (A) Digital images and photographs of osteogenically differentiated 3D printed hMSC construct morphology (B) before and (C) after Alizarin red S staining. Scale bars indicate 5 mm. (D) Digital images and photographs of chondrogenically differentiated 3D printed hMSC construct morphology E) before and (F) after Toluidine blue O staining. The constructs presented well-preserved structures after long-term 4-week culture without evidence of construct deformation due to cellular contraction or proliferation, and generation of specific tissue types (i.e., bone and cartilage) with desired geometries. Scale bar indicates 5 mm. Photomicrographs of (H) Alizarin Red S and (H) Toluidine Blue O stained construct sections. The images demonstrate hMSC differentiation and deposition of lineage specific ECM in the cell-only bioink printed constructs. Scale bars indicate 200 μm.

FIG. 6 illustrates photomicrographs of Safranin-O stained smaller (left) and larger (right) OMA microgels. Scale bars indicate 200 μm.

FIGS. 7(A-F) illustrate plots showing (A) Storage (G′) and loss (G″) moduli of alginate microgel supporting medium (mean microgel diameter=409.6±193.7 μm) as a function of frequency. G′ is larger than G″ over the measured frequency range and both moduli exhibit frequency independence. Viscosity measurements of alginate microgel supporting medium as a function of (B) shear rate and (C) shear strain demonstrate its shear-thinning behavior. (D) G′ and G″ of the alginate microgel supporting medium as a function of shear strain exhibit its shear-yielding behavior and gel-to-sol transition at higher shear strain. (E) Shear moduli and (F) viscosity changes in dynamic strain tests of the alginate microgel supporting medium with alternating low (1%) and high (100%) strains at 1 Hz demonstrate its rapid recovery of strength and viscosity within seconds, which indicates “self-healing” or thixotropic properties.

FIG. 8 illustrates optical images of photocrosslinked and uncrosslinked microgels before and after washing process.

FIG. 9 illustrates optical images of chondrogenically differentiated hMSC constructs before and after Toluidine blue O staining (letter C).

FIGS. 10(A-H) illustrates the quantification of (A) ALP activity, (B) Ca²⁺ and (C) DNA content in the 3D printed hMSC constructs cultured in growth media (GM) and osteogenic media (OM) for 4 weeks. (D) ALP activity and (E) Ca²⁺ normalized by DNA. Quantification of (F) GAG production and (G) DNA content in the 3D printed hMSC constructs cultured in growth media (GM) and chondrogenic media (CM) for 3 weeks. (H) GAG content normalized by DNA. *p<0.05 compared to GM.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

The term “bioactive agent” can refer to any agent capable of modulating a function and/or characteristic of a cell and/or promoting tissue formation, destruction, and/or targeting a specific disease state. Examples of bioactive agents can include, but are not limited to, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III) parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

When used with respect to the bioactive agent, the term “controlled release” is intended to mean that the bioactive agent is released over time in contrast to a bolus type administration in which the entire amount of the bioactive agent is presented to the target at one time. The release will vary as explained below.

The term “gene” or “recombinant gene” refers to a nucleic acid comprising an open reading frame encoding a polypeptide, including both exonic and (optionally) intronic sequences.

The term “gene construct” refers to a vector, plasmid, viral genome or the like which includes an “coding sequence” for a polypeptide or which is otherwise transcribable to a biologically active RNA (e.g., antisense, decoy, ribozyme, etc.), can transfect cells, preferably mammalian cells, and can cause expression of the coding sequence in cells transfected with the construct. The gene construct may include one or more regulatory elements operably linked to the coding sequence, as well as intronic sequences, poly adenylation sites, origins of replication, marker genes, etc.

The term “host cell” or “target cell” refers to a cell transduced with a specified transfer vector. The cell is optionally selected from in vitro cells such as those derived from cell culture, ex vivo cells, such as those derived from an organism, and in vivo cells, such as those in an organism.

The term “incorporated” or “encapsulation,” when used in reference to a bioactive agent or other material and a hydrogel particle, denotes formulating a bioactive agent or other material into a hydrogel particle useful for controlled release of such agent or material. As used herein, those terms contemplate any manner by which a bioactive agent is incorporated into a hydrogel particle, including for example: distributed throughout the matrix, appended to the surface of microparticles, and encapsulated inside the matrix or microparticles. The term “coincorporation” or “coencapsulation” as used herein refers to the incorporation of a bioactive agent in a hydrogel particle and at least another bioactive agent or other material.

The term “modulation” refers to both up regulation (i.e., activation or stimulation) and down regulation (i.e., inhibition or suppression) of a response.

The term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides. Exemplary nucleic acids for use in the subject invention include antisense, decoy molecules, recombinant genes (including transgenes) and the like.

The term “biomaterial” refers to any naturally occurring, naturally derived, or synthetic material or substance which is compatible with biological systems.

The terms “biodegradable” and “bioresorbable” may be used interchangeably and refer to the ability of a material (e.g., a natural polymer or macromer) to be fully resorbed in vivo. “Full” can mean that no significant extracellular fragments remain. The resorption process can involve elimination of the original implant material(s) through the action of body fluids, enzymes, cells, and the like.

The term “gel” includes gels and hydrogels.

The term “function and/or characteristic of a cell” can refer to the modulation, growth, and/or proliferation of at least one cell, such as a progenitor cell and/or differentiated cell, the modulation of the state of differentiation of at least one cell, and/or the induction of a pathway in at least one cell, which directs the cell to grow, proliferate, and/or differentiate along a desired pathway, e.g., leading to a desired cell phenotype, cell migration, angiogenesis, apoptosis, etc.

The term “macromer” can refer to any natural polymer or oligomer.

The term “polynucleotide” can refer to oligonucleotides, nucleotides, or to a fragment of any of these, to DNA or RNA (e.g., mRNA, rRNA, siRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, ribonucleoproteins (e.g., iRNPs). The term can also encompass nucleic acids (i.e., oligonucleotides) containing known analogues of natural nucleotides, as well as nucleic acid-like structures with synthetic backbones.

The term “polypeptide” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term “polypeptide” can also include peptides and polypeptide fragments, motifs and the like, glycosylated polypeptides, and all “mimetic” and “peptidomimetic” polypeptide forms.

The term “cell” can refer to any progenitor cell, such as totipotent stem cells, pluripotent stem cells, and multipotent stem cells, as well as any of their lineage descendant cells, including more differentiated cells. The terms “stem cell” and “progenitor cell” are used interchangeably herein. The cells can derive from embryonic, fetal, or adult tissues. Exemplary progenitor cells can be selected from, but not restricted to, totipotent stem cells, induced pluripotent or progenitor stem cells, pluripotent stem cells, multipotent stem cells, mesenchymal stem cells (MSCs), hematopoietic stem cells, neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, endothelial progenitor cells, and cancer stem cells. Additional exemplary progenitor cells are selected from, but not restricted to, de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

The terms “inhibit,” “silencing,” and “attenuating” can refer to a measurable reduction in expression of a target mRNA (or the corresponding polypeptide or protein) as compared with the expression of the target mRNA (or the corresponding polypeptide or protein) in the absence of an interfering RNA molecule of the present invention. The reduction in expression of the target mRNA (or the corresponding polypeptide or protein) is commonly referred to as “knock-down” and is reported relative to levels present following administration or expression of a non-targeting control RNA.

The term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.), which is to be the recipient of a particular treatment. Typically, the terms “patient” and “subject” are used interchangeably herein in reference to a human subject.

The term “tissue” can refer to an aggregate of cells. “Tissue” is typically an aggregate of cells of the same origin, but may be an aggregate of cells of different origins. The cells can have the substantially same or substantially different function, and may be of the same or different type. “Tissue” can include, but is not limited to, an organ, a part of an organ, bone, cartilage, skin, neuron, axon, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic, or ascite tissue.

Embodiments described herein relate to systems and methods for 3D bioprinting living cells to form scaffold-free 3D cell or tissue constructs. The 3D tissue constructs can be used in regenerative medicine, cell-based technologies, tissue engineering, bioprinting, drug discovery, basic biology, and research applications. Unlike previous 3D bioprinting techniques, which depend on external solid materials for structural maintenance or additional process for prefabrication of cell aggregates, the systems and methods described herein use a self-healing, sheer thinning, crosslinkable, biocompatible hydrogel support medium to provide a structural support role for the printed cell constructs, allowing media provision and long-term culture. Precise maintenance of the structure, for example, mirroring an original CAD file, can also be achieved even after maturation of the tissue by cell proliferation, differentiation and ECM production. The self-healing, sheer thinning, crosslinkable, biocompatible hydrogel support medium can be readily removed or separated from the 3D tissue construct by simple agitation or spontaneous degradation, and the cultured 3D constructs can be readily harvested from the self-healing, sheer thinning, crosslinkable, biocompatible hydrogel support medium without damage. The 3D bioprinting system and methods described herein make it possible to print isolated cells without a biomaterial carrier in the bioink, and can contribute to regenerative medicine by permitting generation of biomimetic cellular condensation-based engineered tissues with defined geometries comprised of multiple cell types with controlled spatial placement.

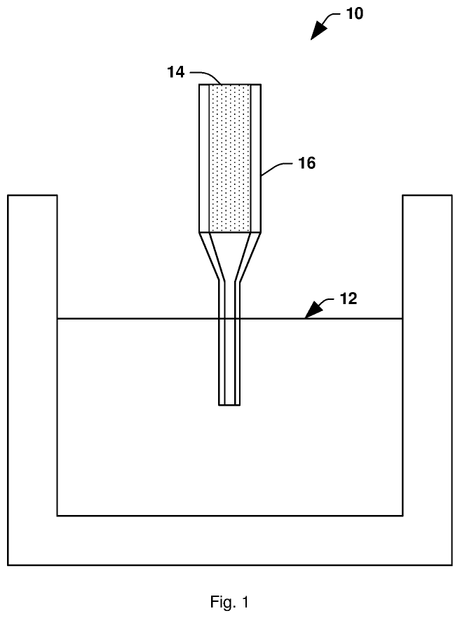

FIG. 1 is a schematic illustration of a system 10 for forming a scaffold-free 3D tissue construct. The system 10 includes a self-healing, shear thinning, crosslinkable, biocompatible hydrogel support medium 12, a first bioink 14, which includes a plurality of cells, and a three dimensional (3D) printer 16 for printing the first bioink 14 in the biocompatible hydrogel support medium 12.

The self-healing, shear thinning, crosslinkable, biocompatible hydrogel support medium can maintain the printed first bioink in a defined shape during printing of the bioink and optionally during culturing of the cells of bioink. The hydrogel support medium can behave as a viscous fluid during printing and be resistant to flow before and after printing. For example, initially, the hydrogel support medium is in a flow-resistant or solid-like state before being printed with the bioink. The hydrogel support medium becomes fluidized under the increased shear stress caused by printing the bioink into the hydrogel support medium. Then, after the printing is finished and the increased shear stress is removed, the hydrogel support medium can self-heal and form a flow-resistant or solid-like stable support medium. The hydrogel support medium can be further crosslinked after printing to maintain the defined shape of the printed first bioink during culturing of cells of the printed bioink.

In some embodiments, the self-healing, shear thinning, crosslinkable, biocompatible hydrogel support medium can include a plurality of crosslinkable hydrogel particles that are provided in a container. The plurality of crosslinkable hydrogel particles are in contact with each other in the container such that interstitial spaces are provided between individual hydrogel particles. The interstitial spaces between the individual particles can form pores in the hydrogel support medium in which a culture medium can be provided and/or flow to the printed bioink during culturing of the cells. The sizes of the pores can be dependent on the sizes of the individual hydrogel particles. For example, smaller pores can result from smaller spaces between the smaller hydrogel particles, and, conversely, larger pores can result from larger spaces between the larger hydrogel particles.

In some embodiments, the hydrogel particles can have an average diameter of about 10 nm to about 10 mm, for example, about 100 nm to about 1000 μm, about 1 μm to about 500 μm, about 25 μm to about 400 μm, or about 50 μm to 200 μm. The plurality of hydrogel particles can have substantially homogenous or similar diameters or include particles of varying diameters to provide a heterogenous mixture of the hydrogel particles.

The hydrogel particles can be cytocompatible and, upon degradation, produce substantially non-toxic products. In some embodiments, the hydrogel particles can include a plurality of crosslinkable biodegradable natural or synthetic polymer macromers. The crosslinkable natural polymer macromers can be any crosslinkable hydrogel forming natural polymer or oligomer that includes a functional group (e.g., a carboxylic group) that can be further polymerized, or ionically linked, or interact via hydrophobic/hydrophilic actions, etc. Examples of natural polymers or oligomers are saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin, and agarose. Other examples include polymer macromers, such as chitosan, PEG, PLGA, PCL and other polymers.

The crosslinkable natural polymer macromers can be at least partially crosslinked using any crosslinking means. For example, the crosslinkable natural polymer macromers can be at least partially crosslinked by ionic crosslinking, chemical crosslinking, photocrosslinking or with the aid of click-reactive groups.

In certain embodiments, the crosslinkable natural or synthetic polymer macromer can include dual crosslinkable natural polymer macromers, such as an acrylated and/or methacrylated natural polymer macromers. Acrylated and/or methacrylated natural polymer macromers can include saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose that can be readily oxidized to form free aldehyde units.

In some embodiments, the acrylated or methacrylated, natural polymer macromers are polysaccharides, which are optionally oxidized so that up to about 50% of the saccharide units therein are converted to aldehyde saccharide units. Control over the degree of oxidation of the natural polymer macromers permits regulation of the gelling time used to form the hydrogel as well as the mechanical properties, which allows for tailoring of the mechanical properties.

In other embodiments, the acrylated and/or methacrylated, natural polymer macromers can include oxidized, acrylated or methacrylated, alginates, which are optionally oxidized so that, for example, up to about 50% of the saccharide units therein are converted to aldehyde saccharide units. Natural source alginates, for example, from seaweed or bacteria, are useful and can be selected to provide side chains with appropriate M (mannuronate) and G (guluronate) units for the ultimate use of the polymer. Alginate materials can be selected with high guluronate content since the guluronate units, as opposed to the mannuronate units, more readily provide sites for oxidation and crosslinking. Isolation of alginate chains from natural sources can be conducted by conventional methods. See Biomaterials: Novel Materials from Biological Sources, ed. Byrum, Alginates chapter (ed. Sutherland), p. 309-331 (1991). Alternatively, synthetically prepared alginates having a selected M and G unit proportion and distribution prepared by synthetic routes, such as those analogous to methods known in the art, can be used. Further, either natural or synthetic source of alginates may be modified to provide M and G units with a modified structure. The M and/or G units may also be modified, for example, with polyalkylene oxide units of varied molecular weight such as shown for modification of polysaccharides in Spaltro (U.S. Pat. No. 5,490,978) with other alcohols such as glycols. Such modification generally will make the polymer more soluble, which generally will result in a less viscous material. Such modifying groups can also enhance the stability of the polymer. Further, modification to provide alkali resistance, for example, as shown by U.S. Pat. No. 2,536,893, can be conducted.

The oxidation of the natural polymer macromers (e.g., alginate material) can be performed using a periodate oxidation agent, such as sodium periodate, to provide at least some of the saccharide units of the natural polymer macromer with aldehyde groups. The degree of oxidation is controllable by the mole equivalent of oxidation agent, e.g., periodate, to saccharide unit. For example, using sodium periodate in an equivalent % of from 2% to 100%, preferably 1% to 50%, a resulting degree of oxidation, i.e., % if saccharide units converted to aldehyde saccharide units, from about 2% to 50% can be obtained. The aldehyde groups provide functional sites for crosslinking and for bonding tissue, cells, prosthetics, grafts, and other material that is desired to be adhered. Further, oxidation of the natural polymer macromer facilitates their degradation in vivo, even if they are not lowered in molecular weight. Thus, high molecular weight alginates, e.g., of up to 300,000 daltons, may be degradable in vivo, when sufficiently oxidized, i.e., preferably at least 5% of the saccharide units are oxidized.

In some embodiments, the natural polymer macromer (e.g., alginate) can be acrylated or methacrylated by reacting an acryl group or methacryl with a natural polymer or oligomer to form the oxidized, acrylated or methacrylated natural polymer macromer (e.g., alginate). For example, oxidized alginate can be dissolved in a solution chemically functionalized with N-hydroxysuccinimide and 1-ethyl-3-(3-dimethylaminopropyl)-carbodiimide hydrochloride to activate the carboxylic acids of alginate and then reacted with 2-amionethylmethacrylate to provide a plurality of methacrylate groups on the alginate.

The degree of acrylation or methacrylation can be controlled to control the degree of subsequent crosslinking of the acrylate and methacrylates as well as the mechanical properties, and biodegradation rate of the hydrogel particles. The degree of acrylation or methacrylation can be about 1% to about 50%, although this ratio can vary more or less depending on the end use of the composition.

In some embodiments, a solution of natural polymer macromers can be ionically crosslinked and/or chemically crosslinked with a first agent to form a plurality of hydrogel particles. The ionically crosslinked hydrogel can be in the form of a plurality of hydrogel particles. By way of example, a solution of natural polymer macromers can be dispensed as microdroplets into an aqueous solution of CaCl₂ and ionically crosslinked to form the plurality of microgels. The extent of crosslinking can be controlled by the concentration of CaCl₂. The higher concentration can correspond to a higher extent of crosslinking. The extent of crosslinking alters the mechanical properties of the hydrogel particles and can be controlled as desired for the particular application. In general, a higher degree of crosslinking results in a stiffer gel.

In some embodiments, the hydrogel particles can be crosslinked with a second agent after being printed with the bioink to form dual crosslinked hydrogel particles. A plurality of second crosslink networks can be formed by crosslinking acrylate and/or methacrylate groups of the acrylated or methacrylated natural polymer macromer. The second crosslinking networks formed by crosslinking the acrylate groups or methacrylate groups of the acrylated and/or methacrylated natural polymer macromer can provide improved mechanical properties, such as resistance to excessive swelling, as well as delayed biodegradation rate of the hydrogel particles.

In some embodiments, the acrylate or methacrylate groups of the acrylated and/or methacrylated natural polymer macromer of the hydrogel particles can be crosslinked by photocrosslinking using UV light or visible light in the presence of photoinitiators. For example, acrylated and/or methacrylated natural polymer macromers of the hydrogel particles can be photocrosslinked with a photoinitiator that is provided in the hydrogel support medium. The hydrogel particles can be exposed to a light source at a wavelength and for a time to promote crosslinking of the acrylate groups of the polymers and form the photocrosslinked biodegradable hydrogel particles.

A photoinitiator can include any photo-initiator that can initiate or induce polymerization of the acrylate or methacrylate macromer. Examples of the photoinitiator can include camphorquinone, benzoin methyl ether, 2-hydroxy-2-methyl-1-phenyl-1-propanone, diphenyl(2,4,6-trimethylbenzoyl)phosphine oxide, benzoin ethyl ether, benzophenone, 9,10-anthraquinone, ethyl-4-N,N-dimethylaminobenzoate, diphenyliodonium chloride and derivatives thereof.

In other embodiments, the hydrogel support medium can further include at least one bioactive agent that is provided in the hydrogel particles or potentially a culture medium that can be added to the hydrogel support medium during culturing of the printed bioink. The bioactive agent can include polynucleotides and/or polypeptides encoding or comprising, for example, transcription factors, differentiation factors, growth factors, and combinations thereof. The at least one bioactive agent can also include any agent capable of modulating a function and/or characteristic of a cell and/or promoting tissue formation (e.g., bone and/or cartilage), destruction, and/or targeting a specific disease state (e.g., cancer). Examples of bioactive agents include chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., EGF), HGF, VEGF, fibroblast growth factors (e.g., bFGF), PDGF, insulin-like growth factor (e.g., IGF-I, IGF-II) and transforming growth factors (e.g., TGF-β I-III) parathyroid hormone, parathyroid hormone related peptide, bone morphogenic proteins (e.g., BMP-2, BMP-4, BMP-6, BMP-7, BMP-12, BMP-13, BMP-14), sonic hedgehog, growth differentiation factors (e.g., GDF5, GDF6, GDF8), recombinant human growth factors (e.g., MP-52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparin sulfate, polynucleotides, DNA fragments, DNA plasmids, MMPs, TIMPs, interfering RNA molecules, such as siRNAs, miRNAs, DNA encoding for an shRNA of interest, oligonucleotides, proteoglycans, glycoproteins, and glycosaminoglycans.

In some embodiments, a bioactive agent can comprise an interfering RNA or miRNA molecule incorporated on or within insoluble native collagen fibers or dispersed on or within the cell aggregate. The interfering RNA or miRNA molecule can include any RNA molecule that is capable of silencing an mRNA and thereby reducing or inhibiting expression of a polypeptide encoded by the target mRNA. Alternatively, the interfering RNA molecule can include a DNA molecule encoding for a shRNA of interest. For example, the interfering RNA molecule can comprise a short interfering RNA (siRNA) or microRNA molecule capable of silencing a target mRNA that encodes any one or combination of the polypeptides or proteins described above.

The first bioink, which is capable of being printed with the 3D printer into the hydrogel support medium, includes a plurality of cells and optionally a macromer carrier, nanoparticles, microparticles, bioactive agents, cell aggregates, and/or organoids. The cells provided in the first bioink, whether individual cells or in the form of a cell aggregate or organoid, can be autologous, xenogeneic, allogeneic, and/or syngeneic. Where the cells are not autologous, it may be desirable to administer immunosuppressive agents in order to minimize immunorejection. The cells employed may be primary cells, expanded cells, or cell lines, stem cells, iPSCs, embryonic cells, and may be dividing or non-dividing cells. For example, autologous cells can be expanded in this manner if a sufficient number of viable cells cannot be harvested from the host subject. Alternatively or additionally, the cells may be cell aggregates, pieces of tissue, including tissue that has some internal structure, or organoids. The cells may be primary tissue explants and preparations thereof, cell lines (including transformed cells).

In some embodiments, the cells can be an undifferentiated or substantially differentiated progenitor cells. In other embodiments, the progenitor cell can be an adult stem cell or adult cancer cell. The stem cell, such as an adult stem cell or adult cancer cell, can be isolated from animal or human tissues. In some embodiments described herein, the stem cell can be isolated from, but not limited to, tendon/ligament tissue, bone morrow, adipose tissue or dental pulp. The bioink can include at least about 10%, at least about 20%, at least about 30%, at least about 40%, at least about 50%, at least about 60%, at least about 70%, at least about 80% cells, at least about 90% cells, at least about 95% cells, at least about 99% cells based on the total volume of the bioink.

In other embodiments, the first bioink can be free of or substantially free of the optional macromer carrier, nanoparticles, microparticles, bioactive agents, cell aggregates, and/or organoids. The first bioink can be in a liquid or slurry form during printing.

In some embodiments, the bioink can include at least one, two, three, or more bioactive agent(s) as described herein that can be capable of modulating a function and/or characteristic of a cell. For example, the bioactive agent may be capable of modulating a function and/or characteristic of a cell in the bioink.

In other embodiments, the bioink can further include various nanoparticles and/or microparticles dispersed with the cells in the first bioink. The nanoparticles and/or microparticles that are dispersed in the bioink can be formed from a biocompatible and biodegradable material that is capable of improving properties of the cells in the bioink and can upon degradation be substantially non-toxic. The microparticles can have a diameter less than 1 mm and typically between about 1 nm and about 200 μm, e.g., about 20 μm to about 100 μm. The nanoparticles and/or microparticles can include nanospheres, nanocapsules, microspheres, and microcapsules, and may have an approximately spherical geometry and be of fairly uniform size.

The nanoparticles and/or microparticles can include nanospheres and/or microspheres that have a homogeneous composition as well as nanocapsules and/or microcapsules, which include a core composition (e.g., a bioactive agent) distinct from a surrounding shell. For the purposes of this disclosure, the terms “nanosphere,” “nanoparticle,” and “nanocapsule” may be used interchangeably, and the terms “microsphere,” “microparticle,” and “microcapsule” may be used interchangeably.

In some embodiments, the nanoparticles and/or microparticles can be formed from a biocompatible and biodegradable polymer. Examples of biocompatible, biodegradable polymers include natural polymers, such fibrin, gelatin, glycosaminoglycans (GAG), poly (hyaluronic acid), poly(sodium alginate), alginate, hyaluronan, and agarose. Other examples of biocompatible, biodegradable polymers are poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, and blends and/or copolymers thereof.

Still other examples of materials that may be used to form nanoparticles and/or microparticles can include chitosan, poly(ethylene oxide), poly (lactic acid), poly(acrylic acid), poly(vinyl alcohol), poly(urethane), poly(N-isopropyl acrylamide), poly(vinyl pyrrolidone) (PVP), poly (methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), polyhydroxybutyrate (PHB), copolymers thereof, and blends thereof.

The nanoparticles and/or microparticles can also be formed from inorganic materials, such as calcium phosphate materials including mineralite, carbonated nano-apatite, calcium phosphate based mineralite, tri-calcium phosphate, octa-calcium phosphate, calcium deficient apatite, amorphous calcium phosphate, hydroxyapatite, substitute apatite, carbonated apatite-like minerals, highly substituted carbonated apatites or a mixture thereof. Calcium phosphate nanoparticles and/or microparticles can have an average particle size of between about 1 nm and about 200 μm. It will be appreciated that smaller or larger calcium phosphate nanoparticles and/or microparticles may be used. The calcium phosphate nanoparticles and/or microparticles can have a generally spherical morphology and be of a substantially uniform size or, alternatively, may be irregular in morphology. Calcium phosphate nanoparticles and/or microparticles may be complexed with surface modifying agents to provide a threshold surface energy sufficient to bind material (e.g., bioactive agents) to the surface of the microparticle without denaturing the material. Non-limiting examples of surface modifying agents can include basic or modified sugars, such as cellobiose, carbohydrates, carbohydrate derivatives, macromolecules with carbohydrate-like components characterized by an abundance of —OH side groups and polyethylene glycol.

It will be appreciated at least one bioactive agent can also be incorporated on or encapsulated within the nanoparticles and/or microparticles. The nanoparticles and/or microparticles can differentially or controllably release the at least one bioactive agent or be taken up by at least one cell to modulate the function and/or characteristic of the cell. The at least one bioactive agent may be at least partially coated on the surface of the at least one of the nanoparticles and/or microparticles. Alternatively, the at least one bioactive agent may be dispersed, incorporated, and/or impregnated within the nanoparticles and/or microparticles.

In some embodiments, the bioink can include a plurality of first nanoparticles and/or microparticles that can include or release one or more first bioactive agent(s) and a plurality of second nanoparticles and/or microparticles that can include or release one or more second bioactive agent(s). The one or more first bioactive agents and the one or more second bioactive agents may comprise the same or different agents. The one or more first bioactive agents and the one or more second bioactive agents can be differentially, sequentially, and/or controllably released from the first nanoparticles and/or microparticles and second nanoparticles and/or microparticles to modulate a different function and/or characteristic of a cell. It will be appreciated that the one or more first bioactive agents can have a release profile that is the same or different from the release profile of the one or more second bioactive agents from the first nanoparticles and/or microparticles and the second nanoparticles and/or microparticles. Additionally, it will be appreciated that the first nanoparticles and/or microparticles can degrade or diffuse before the degradation or diffusion of the second nanoparticles and/or microparticles or allow for an increased rate of release or diffusion of the one or more first bioactive agents compared to the release of the one or more second bioactive agents. The first and second nanoparticles and/or microparticles may be dispersed uniformly within the bioink or, alternatively, dispersed such that different densities of the first nanoparticles and/or microparticles and second nanoparticles and/or microparticles are provided in different bioinks or in different portions of a cell aggregate formed using different or multiple bioinks.

The macromer carrier provided in the bioink can potentially include any natural polymer macromers. Examples of natural polymers or oligomers are saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose.

In some embodiments, the bioink can additionally include non-cellular materials that provide specific mechanical properties that enhance the ability to bioprinting.

In other embodiments, the system can further include an additional bioink, such as a second, third, fourth, or more bioink, that is capable of being printed with the 3D printer into the hydrogel support medium. The additional (second, third, fourth, or more) bioinks can be different from the first bioink and include different types cells, cell aggregates, macromer solution, nanoparticles, microparticles, bioactive agents, cell aggregates, organoids as well as different concentrations of cells, cell aggregates, macromer solution, nanoparticles, microparticles, bioactive agents, cell aggregates, organoids, and/or combinations thereof. The second bioink can be printed before, during, or after printing in the hydrogel support medium with the first bioink.

In some embodiments, the 3D printer, which prints the first bioink and optional additional bioink in the hydrogel support medium can include any printer that provides three-dimensional, precise deposition of the bioink via, for example, a methodology that is compatible with an automated, computer-aided, three-dimensional prototyping device.

In some embodiments, the 3D printer is a bioprinter that can dispense the bioink from a cartridge or container in a specific pattern and at specific positions in the hydrogel support medium as directed by a computer aided design (CAD) software in order to form a specific cellular construct, tissue, or organ. In order to fabricate complex tissue constructs, the bioprinter deposits the bioink at precise speeds and in uniform amounts. In some embodiments, a cartridge containing the bioink comprises one dispensing orifice. In various other embodiments, a cartridge comprises 1, 2, 3, 4, 5, 6, 7, 8, 9, 10, 11, 12, 13, 14, 15, 20, 30, 40, 50, 60, 70, 80, 90, 100, or more dispensing orifices. In further embodiment, the edges of a dispensing orifice are smooth or substantially smooth.

Many types of cartridges are suitable for use with bioprinters. In some embodiments, a cartridge is compatible with bioprinting that involves extruding the bioink, which includes the cells, through one or more dispensing orifices. In some embodiments, a cartridge is compatible with non-continuous bioprinting. In some embodiments, a cartridge is compatible with continuous and/or substantially continuous bioprinting.

In some embodiments, a cartridge is a capillary tube or a micropipette. In other embodiments, a cartridge is a syringe or a needle. Many internal diameters are suitable for substantially round or cylindrical cartridges. In various embodiments, suitable internal diameters include, by way of non-limiting examples, 1 μm, 10 μm, 50 μm, 100 μm, 200 μm, 300 μm, 400 μm, 500 μm, 600 μm, 700 μm, 800 μm, 900 μm, 1000 μm or more, including increments therein.

In some embodiments, a cartridge has an internal diameter of about 1 μm to about 5 mm. In a particular embodiment, a cartridge has an internal diameter of about 500 μm. In another particular embodiment, a cartridge has an internal diameter of about 250 μm. Many internal volumes are suitable for the cartridges disclosed herein. In various embodiments, suitable internal volumes include, by way of non-limiting examples, 0.1 ml, 1 ml, 10 ml, 20 ml, 30 ml, 40 ml, 50 ml, 100 ml, 200 ml, 300 ml, 400 ml, 500 ml, 600 ml, 700 ml, 800 ml, 900 ml, 1000 ml or more, including increments therein.

The system described herein can further include a culture medium for modulating a function or characteristic of a cell and/or for promoting growth and/or differentiation of the cells of the printed bioink. For example, the culture medium can include a cell differentiation medium, such as an osteogenic differentiation media or chondrogenic differentiation media. The hydrogel support medium containing the printed bioink can be provided in the culture medium after crosslinking or before further crosslinking the hydrogel support medium to enhance the stability of the hydrogel support medium.

It will be appreciated that growth factors can be added to the medium to enhance or stimulate cell growth. Examples of growth factors include transforming growth factor-β (TGF-β) (e.g., TGF-β1 or TGF-β3), platelet-derived growth factor, insulin-like growth factor, acid fibroblast growth factor, basic fibroblast growth factor, epidermal growth factor, hepatocytic growth factor, keratinocyte growth factor, and bone morphogenic protein. It will also be appreciated that other agents, such as cytokines, hormones (e.g., parathyroid hormone, parathyroid hormone-related protein, hydrocortisone, thyroxine, insulin), fatty acids (e.g., Omega-3 fatty acids such as α18:3 linolenate), and/or vitamins (e.g., vitamin D), may also be added or removed from the culture medium to promote cell growth.

The system, described herein, can be used in a method for forming a scaffold-free 3D tissue construct. The method can include providing the self-healing, shear thinning, crosslinkable, biocompatible hydrogel support medium. The first bioink can be printed into the hydrogel support medium in a defined shape using the 3D printer. The 3D printer can dispense the bioink in a specific pattern and at specific positions in the hydrogel support medium as directed by CAD software in order to form a 3D printed structure with a defined shape. The first bioink can include a plurality of cells and optional macromer carrier, nanoparticles, microparticles, bioactive agents, cell aggregates, and/or organoids.

The hydrogel support medium can maintain the printed bioink in the defined shape during printing. The hydrogel support medium can be in a flow-resistant or solid-like state before being printed with the bioink. The hydrogel support medium becomes fluidized under the increased shear stress caused by printing the bioink into the hydrogel support medium. Then, after the printing is finished and the increased shear stress is removed, the hydrogel support medium can self-heal and form a flow-resistant or solid-like stable support medium.

The hydrogel support medium can be further crosslinked after printing to maintain the defined shape of the printed first bioink during culturing of the cells in the hydrogel support medium. The crosslinking of the hydrogel support medium can be dependent on the crosslinkable natural or synthetic polymer macromers used or any crosslinking agent added to hydrogel support medium. As described herein, the hydrogel support medium can be ionically, chemically or photocrosslinked as well as crosslinked using click-reactive natural polymer macromers, or by any crosslinking means.

After further crosslinking the hydrogel support medium including the printed bioink, the hydrogel support medium can be provided in or transferred to a culture medium bath modulating a function or characteristic of a cell and/or for promoting growth and/or differentiation of the cells of the printed bioink. For example, the culture medium can include a cell differentiation medium, such as an osteogenic differentiation media or chondrogenic differentiation media. Culturing in the hydrogel support medium, which includes the printed bioink, in a culture medium after crosslinking the hydrogel support medium can maintain the printed bioink in the defined shape and allow the printed bioink to form cell aggregate or tissue construct structure with the defined shape.

The crosslinked hydrogel support medium can be readily removed or separated from the cell aggregate or tissue construct after culturing by, for example, simple agitation or spontaneous degradation of the hydrogel support medium. The cell aggregate or tissue constructs can be readily harvested from the self-hydrogel support medium without damage.

In some embodiments, the method can include printing an additional bioink, such as a second, third, fourth, or more bioink, into the hydrogel support medium. The additional bioink is different than the first bioink and includes a plurality of cells, cell aggregates, a macromer carrier, nanoparticles, microparticles, bioactive agents, organoids, and/or combinations thereof.

In some embodiments, a construct and/or tissue construct so formed from the printed bioink can be used to promote tissue growth in a subject. For example, a target site for the tissue construct can be identified in a subject in need thereof. The target site can comprise a tissue defect (e.g., cartilage and/or bone defect) in which promotion of new tissue (e.g., cartilage and/or bone) is desired. The target site can also comprise a diseased location (e.g., tumor). Methods for identifying tissue defects and disease locations are known in the art and can include, for example, various imaging modalities, such as CT, MRI, and X-ray.

In some embodiments, the tissue defect can include a defect caused by the destruction of bone or cartilage. For example, one type of cartilage defect can include a joint surface defect. Joint surface defects can be the result of a physical injury to one or more joints or, alternatively, a result of genetic or environmental factors. Most frequently, but not exclusively, such a defect will occur in the knee and will be caused by trauma, ligamentous instability, malalignment of the extremity, meniscectomy, failed ACI or mosaicplasty procedures, primary osteochondritis dessecans, osteoarthritis (early osteoarthritis or unicompartimental osteochondral defects), or tissue removal (e.g., due to cancer). Examples of bone defects can include any structural and/or functional skeletal abnormalities. Non-limiting examples of bone defects can include those associated with vertebral body or disc injury/destruction, spinal fusion, injured meniscus, avascular necrosis, cranio-facial repair/reconstruction (including dental repair/reconstruction), osteoarthritis, osteosclerosis, osteoporosis, implant fixation, trauma, and other inheritable or acquired bone disorders and diseases.

Tissue defects can also include cartilage defects. Where a tissue defect comprises a cartilage defect, the cartilage defect may also be referred to as an osteochondral defect when there is damage to articular cartilage and underlying (subchondral) bone. Usually, osteochondral defects appear on specific weight-bearing spots at the ends of the thighbone, shinbone, and the back of the kneecap. Cartilage defects in the context of the present invention should also be understood to comprise those conditions where surgical repair of cartilage is required, such as cosmetic surgery (e.g., nose, ear). Thus, cartilage defects can occur anywhere in the body where cartilage formation is disrupted, where cartilage is damaged or non-existent due to a genetic defect, where cartilage is important for the structure or functioning of an organ (e.g., structures such as menisci, the ear, the nose, the larynx, the trachea, the bronchi, structures of the heart valves, part of the costae, synchondroses, enthuses, etc.), and/or where cartilage is removed due to cancer, for example.

After identifying a target site, the tissue construct formed from the composition or bioink described herein having a desired shape can be administered to the target site. The tissue construct can be prepared according to the method described above.

The following example is for the purpose of illustration only and is not intended to limit the scope of the claims, which are appended hereto

### EXAMPLE

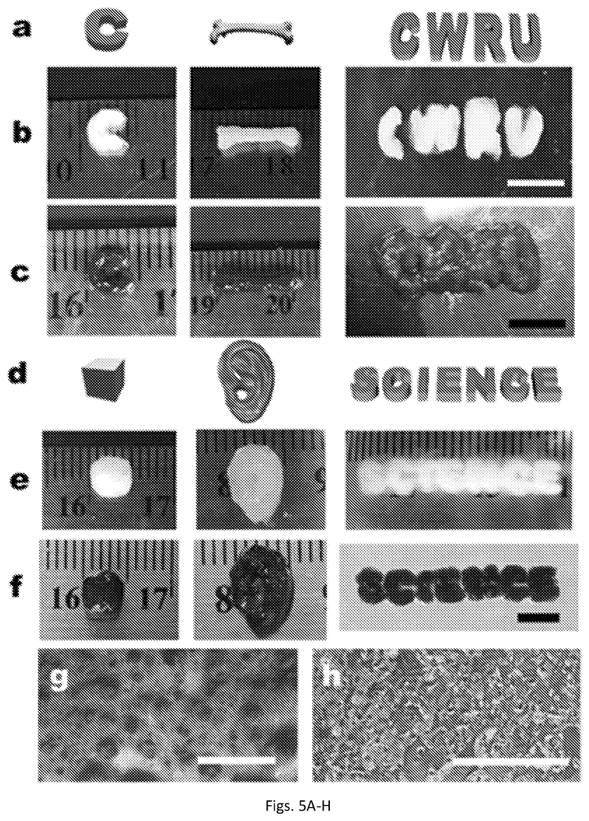

In this Example, we present newly generated tissues from directly assembled stem cells, which have been 3D bioprinted into a photo-curable liquid-like supporting medium comprised of solid hydrogel microparticles (microgels) (FIG. 5). The supporting bath consists of biodegradable and photocrosslinkable alginate microgels, which are prepared by ionic crosslinking of dual-crosslinkable, oxidized and methacrylated alginate (OMA), and is expected to applicable to general 3D bioprinting systems. The microgel supporting medium sustains the high-resolution printing of human bone marrow-derived mesenchymal stem cells (hMSCs) by exhibiting similar properties to Bingham plastic fluids. While the microgel supporting medium allows the printing needle move freely via its shear-thinning properties, the microgels work as supporting materials for printed constructs through self-healing properties. After directly 3D bioprinting of hMSCs into the microgel supporting medium, photocrosslinking of the microgels can provide mechanical stability for hMSC constructs for long-term culture. Dissociation of the photocrosslinked microgel supporting medium by gentle agitation may facilitate acquisition of matured 3D tissue constructs. Collectively, our objectives were (i) to assess the effect of the size of dual-crosslinkable OMA microgels in the supporting bath on printing resolution, (ii) to evaluate the capacity of the OMA supporting bath to maintain the viability individual printed cells and the structure of resulting self-assembly printed constructs, and (iii) to investigate function of the obtained 3D scaffold-free cellular constructs.

### Materials and Methods

### Synthesis of OMA

Oxidized alginate (OA) was prepared by reacting sodium alginate with sodium periodate. Sodium alginate (10 g, Protanal LF 200S, FMC Biopolymer) was dissolved in ultrapure deionized water (diH₂O, 900 ml) overnight. Sodium periodate (0.1 g, Sigma) was dissolved in 100 ml diH₂O, added to alginate solution under stirring to achieve 1% theoretical alginate oxidation, and allowed to react in the dark at room temperature for 24 hrs. The oxidized, methacrylated alginate (OMA) macromer was prepared by reacting OA with 2-aminoethyl methacrylate (AEMA). To synthesize OMA, 2-morpholinoethanesulfonic acid (MES, 19.52 g, Sigma) and NaCl (17.53 g) were directly added to an OA solution (1 L) and the pH was adjusted to 6.5. N-hydroxysuccinimide (NHS, 1.176 g, Sigma) and 1-ethyl-3-(3-dimethylaminopropyl)-carbodiimide hydrochloride (EDC, 3.888 g, Sigma) were added to the mixture under stirring to activate 20% of the carboxylic acid groups of the alginate. After 5 min, AEMA (1.688 g, Polysciences) (molar ratio of NHS:EDC:AEMA=1:2:1) was added to the solution, and the reaction was maintained in the dark at RT for 24 hrs. The reaction mixture was precipitated into excess of acetone, dried in a fume hood, and rehydrated to a 1% w/v solution in diH₂O for further purification. The OMA was purified by dialysis against diH₂O using a dialysis membrane (MWCO 3500, Spectrum Laboratories Inc.) for 3 days, treated with activated charcoal (5 g/L, 50-200 mesh, Fisher) for 30 min, filtered (0.22 μm filter) and lyophilized. To determine the levels of alginate methacrylation, the OMA was dissolved in deuterium oxide (D₂O, 2 w/v %), and ¹H-NMR spectra were recorded on a Varian Unity-300 (300 MHz) NMR spectrometer (Varian Inc.) using 3-(trimethylsilyl)propionic acid-d₄ sodium salt (0.05 w/v %) as an internal standard.

### Fabrication of OMA Microgel Slurry

To fabricate smaller OMA microgels, OMA (1.5 g) was dissolved in DMEM (100 ml) containing 0.05% photoinitiator (PI), placed in a syringe, and then dispensed into a gelling bath containing an aqueous solution of CaCl₂ (1 L, 0.2 M) bath. After fully ionically crosslinking the OMA fibers in the bath for 30 min, the resultant OMA fibers were collected, washed with DMEM three times, and then blended using a consumer-grade blender (Osterizer MFG, at “pulse” speed) for 90 sec with 100 ml DMEM. Then, the blended OMA slurry was loaded into 50 ml conical tubes and centrifuged at 2000×g for 5 min. The supernatant was removed and replaced with a sterile 70% ethanol. The slurry was vortexed back into suspension and centrifuged again. After the supernatant was removed, the OMA microgel slurry was vortexed with sterile 70% ethanol and then stored until use at 4° C. To fabricate larger sized OMA micogels, OMA solution was loaded into a 3-ml syringe, and then the syringe was connected to a custom coaxial microdroplet generator designed in our laboratory. The OMA solution was pumped at 0.5 ml/sec with an outer air flow rate of 10 L/min, and the droplets dripped into a collection bath containing an aqueous solution of CaCl₂ (0.2 M). After fully ionically crosslinking the microgels in the bath for 30 min, the resultant OMA microgels were collected and washed with DMEM three times. The OMA microgels were suspened in sterile 70% ethanol and stored until use at 4° C.

To evaluate the morphology and measure the size of OMA microgels comprising the slurries, the slurries were centrifuged at 2000×g for 5 min The supernatants were removed and replaced with DMEM containing 0.05% PI, and the microgels were vortexed back into suspension and then centrifuged again. This process was repeated five times and then the supernatants were removed. To visualize the OMA microgels, they were stained with Safranin O and then imaged using a microscope (Leitz Laborlus S, Leica) equipped with a digital camera (Coolpix 995, Nikon). To measure the mean diameter of the smaller OMA microgels (prepared using a blender), 1 ml of the OMA microgels were suspended in 10 ml DMEM containing 0.05% PI and measured at room temperature by dynamic light scattering using a particle size analyzer (90Plus, Brookhaven Instruments). The mean diameter of the larger OMA microgels (prepared via the coaxial microdroplet generator) was measured using ImageJ with the images of Safranin O stained OMA microgels.

### Rheological Properties of OMA Microgel Slurry

Dynamic rheological examination of the OMA microgel slurries was performed to evaluate shear-thinning, self-healing and mechanical properties with a Haake MARS III rotational rheometer (ThermoFisher Scientific). In oscillatory mode, a parallel plate (80 mm diameter) geometry measuring system was employed, and the gap was set to 1 mm. After each OMA microgel slurry was placed between the plates, all the tests were started at 37±0.1° C., and the plate temperature was maintained at 37° C. Oscillatory frequency sweep (0.01-1.3 Hz at 1% strain) tests were performed to measure storage moduli (G′), loss moduli (G″) and viscosity. Oscillatory strain sweep (0.1-100% strain at 1 Hz) tests were performed to show the shear-thinning characteristics of the OMA microgels and to determine the shear-yielding points at which the OMA microgel slurries behave fluid-like. To demonstrate the self-healing properties of OMA microgel slurries, cyclic deformation tests were performed at 100% strain with recovery at 1% strain, each for 1 min at 1 Hz.

Preparation of hMSC Ink

To isolate human bone marrow-derived mesenchymal stem cells (hMSCs), bone marrow aspirates were obtained from the posterior iliac crest of a healthy twenty seven-year old male donor under a protocol approved by the University Hospitals of Cleveland Institutional Review Board. The aspirates were washed with growth medium comprised of low-glucose Dulbecco's Modified Eagle's Medium (DMEM-LG, Sigma) with 10% prescreened fetal bovine serum (FBS, Gibco). Mononuclear cells were isolated by centrifugation in a Percoll (Sigma) density gradient and the isolated cells were plated at 1.8×10⁵ cells/cm² in DMEM-LG containing 10% FBS and 1% penicillin/streptomycin (P/S, Thermo Fisher Scientific) in an incubator at 37° C. and 5% CO₂. After 4 days of incubation, non-adherent cells were removed and adherent cell were maintained in DMEM-LG containing 10% FBS, 1 P/S and 10 ng/ml FGF-2 with media changes every 3 days. After 14 days of culture, the cells were passaged at a density of 5×10³ cells/cm², cultured for an additional 14 days, and then stored in cryopreservation media in liquid nitrogen until use. To use hMSC as a bioink, hMSCs were expanded in growth media consisting of DMEM-LG with 10% FBS (Sigma), 1% P/S and 10 ng/ml FGF-2 and loaded into a 2.5-ml syringe (Gastight Syringe, Hamilton Company).

Modification of 3D Printer

All 3D printing was performed using a 3D printer (PrintrBot Simple Metal 3D Printer, Printrbot) modified with a syringe-based extruder. The stock thermoplastic extruder assembly was replaced with a custom-built syringe pump extruder. The syringe pump extruder was designed to use the NEMA-17 stepper motor from the original Printrbot thermoplastic extruder and mount directly in place of the extruder on the x-axis carriage. The syringe pump extruder was printed with polylactic acid using the thermoplastic extruder on the Printrbot before its removal. Using the same stepper motor, the syringe pump extruder was natively supported by the software that came with the printer. The design for the syringe pump extruder and the image file of the human femur were downloaded as STL files from the NIH 3D Print Exchange (http://3dprint.nih.gov) under open-source license. Digital image files of letters for 3D printing were generated from www.tinkercad.com. The file for the human ear was downloaded from www.thinkiver.com/thing:304657 under the terms of the Creative Commons Attribution license, which permits unrestricted use, reproduction and distribution in any medium.

3D Printing of hMSCs

The hMSC-loaded syringe was connected to a 0.5-inch 22G stainless steel needle (McMaster-Carr) and mounted into the syringe pump extruder on the 3D printer. A petri dish was filled with OMA microgel slurry at room temperature to serve as a supporting bath and placed on the building platform. The tip of the needle was positioned at the center and near the bottom of the dish, and the print instructions were sent to the printer using the host software (Cura Software, Ultimaker), which is an open source 3D printer host software. After 3D printing of hMSCs, OMA microgel supporting medium with a 3D printed construct was stabilized by photocros slinking under UV at 20 mW/cm² for 1 min. After slurry photocrosslinking, 3D printed hMSC constructs in the photocrosslinked OMA microgel slurry were transferred into 6-well tissue culture plates with growth media, chondrogenic differentiation media or osteogenic differentiation media, and placed in a humidified incubator at 37° C. with 5% CO₂.

Analysis of Printed hMSC Structures

Linear hMSC filaments were printed in the OMA microgel supporting baths with 22, 25 and 27 G needles, baths were photocrosslinked under UV light a 20 mW/cm² for 1 mM, and then 5 ml culture media was added. The viability and morphology of 3D printed hMSC filaments were investigated using a Live/Dead staining comprised of fluorescein diacetate [FDA, 1.5 mg/ml in dimethyl sulfoxide (Research Organic Inc.), Sigma] and ethidium bromide (EB, 1 mg/ml in PBS, Thermo Fisher Scientific). The staining solution was freshly prepared by mixing 1 ml FDA solution and 0.5 ml EB solution with 0.3 ml PBS (pH 8). 100 μl of staining solution was added into each well and incubated for 10 min at room temperature, and then stained 3D printed hMSC filaments were imaged using a fluorescence microscope (ECLIPSE TE 300) equipped with a digital camera (Retiga-SRV). Diameters of the 3D printed hMSC filaments were measured at least 400 times for each group using ImageJ (National Institutes of Health)

Osteogenic and Chondrogenic Differentiation of the 3D Printed hMSC Constructs

3D printed hMSC constructs in the photocrosslinked OMA microgel supporting baths were differentiated by culture with osteogenic differentiation media [10 mM β-glycerophosphate (CalBiochem), 37.5 μg/ml ascorbic acid (Wako USA), 100 nM dexamethasone (MP Biomedicals), and 100 ng/ml BMP-2 in DMEM-high glucose] containing 10% FBS and 1% P/S or chondrogenic differentiation media [1% ITS+Premix, 100 nM dexamethasone, 37.5 μg/ml 1-ascorbic acid-2-phosphate, 1 mM sodium pyruvate, 100 μM nonessential amino acids, and 10 ng/ml TGF-β₁ in DMEM-high glucose]. The osteogenic and chondrogenic media was changed twice a week. After 4 weeks of culture in osteogenic differentiation media, 3D printed hMSC constructs were fixed in 10% neutral buffered formalin overnight at 4° C. and stained with Alizarin red S. Cryosectioned samples were also stained with Alizarin red S. After 3 weeks of culture in chondrogenic differentiation media, 3D printed hMSC constructs were fixed in 10% neutral buffered formalin over night at 4° C. and stained with Toluidine blue O. Cryosectioned samples were also stained with Toluidine blue O. For quantification of alkaline phosphatase (ALP) activity, DNA content and calcium deposition, osteogenically differentiated 3D printed hMSC constructs were homogenized at 35,000 rpm for 30 s using a TH homogenizer (Omni International) in 1 ml ALP lysis buffer (CelLytic™ M, Sigma). The homogenized solutions were centrifuged at 500 g with a Sorvall Legent RT Plus Centrifuge (Thermo Fisher Scientific). For ALP activity measurements, supernatant (100 μ1) was treated with p-nitrophenylphosphate ALP substrate (pNPP, 100 μl, Sigma) at 37° C. for 30 min, and then 0.1 N NaOH (50 μl) was added to stop the reaction. The absorbance was measured at 405 nm using a plate reader (FMAX, Molecular Devices) (N=4). A standard curve was made using the known concentrations of 4-nitrophenol (Sigma). DNA content in supernatant (100 μl) was measured using a Quant-iT PicoGreen assay kit (Invitrogen) according to the manufacturer's instructions. Fluorescence intensity of the dye-conjugated DNA solution was measured using a plate reader (FMAX) set at 485 nm excitation and 538 nm emission (N=4). After an equal volume of 1.2 N HCl was added into each lysate solution, the mixed solutions were centrifuged at 500 g with a Sorvall Legent RT Plus Centrifuge. Calcium deposition of the constructs was quantified using a calcium assay kit (Pointe Scientific) according to the manufacturer's instructions. Supernatant (4 μl) was mixed with a color and buffer reagent mixture (250 μl) and the absorbance was read at 570 nm on a plate reader (FMAX, N=4). All ALP activity and calcium deposition measurements were normalized to DNA content. To measure GAG production, chondrogenically differentiated 3D printed hMSC constructs were digested in papain buffer (1 mL, pH 6.5)) containing papain (25 μg mil, Sigma), 1-cysteine (2×10⁻³M, Sigma), sodium phosphate (50×10⁻³M) and EDTA (2×10⁻³M) at 65° C. overnight. GAG content (N=4) was quantified by a dimethylmethylene blue assay⁴ and DNA content (N=4) was measured using the PicoGreen assay as described above. GAG content was also normalized to DNA content. Quantitative data were expressed as mean±standard deviation. Statistical analysis was performed with unpaired Student t-test using Graphpad Prism (GraphPad). A value of p<0.05 was considered statically significant.

### Results

3D Bioprinting of Living hMSCs Without a Biomaterial in the Bioink

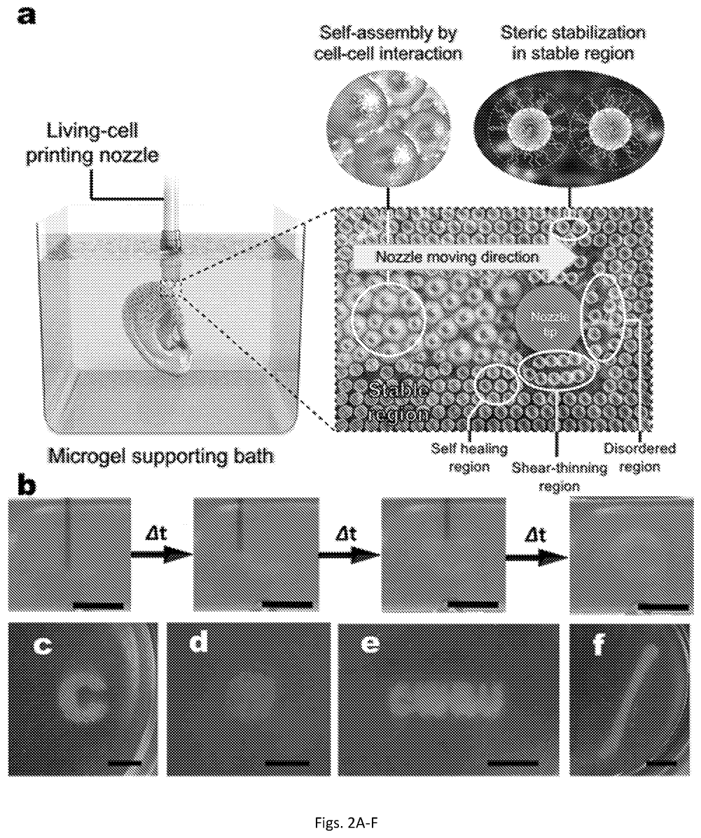

Living hMSCs can be printed as a bioink by themselves without a carrier macromer solution into a photo-curable, self-healing and shear-thinning alginate microgel supporting medium, which is formed with calcium-crosslinked OMA microgels (FIG. 2). Alginate microgel supporting medium is fluidized under low shear stress, permitting easy insertion and rapid motion of needles deep within the bulk. After removing shear stress caused by needle movement and ejection of printing material, the locally fluidized alginate microgel bath rapidly “self-heals” and forms a stable medium that firmly holds the printed hMSCs in 3D place (FIG. 2A). The low yield stress of the alginate microgel medium in its solid state and its rapid self-healing behavior allows the unrestricted deposition, placement and structuring of cells deep within the alginate microgel supporting medium that maintains the printed structure with fidelity (FIG. 2B). To explore the versatility and stability of 3D printing into the alginate microgel supporting medium, a variety of complicated 3D structures were printed using only individual cells as a bioink. A letter (C), an ear, letters comprising an acronym (CWRU) and a femur were successfully created with high resolution (FIGS. 2C-F).

### Properties of the Alginate Microgel Supporting Medium

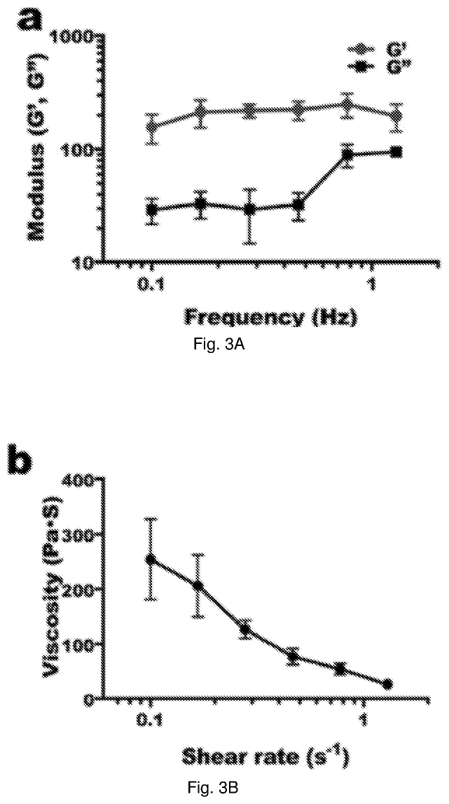

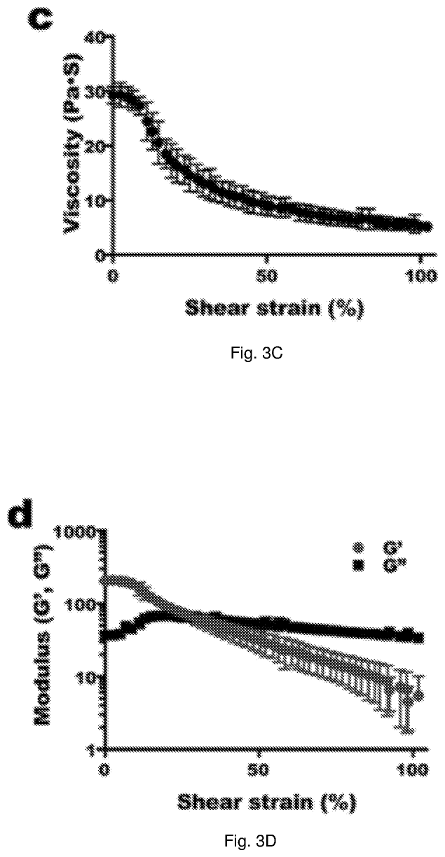

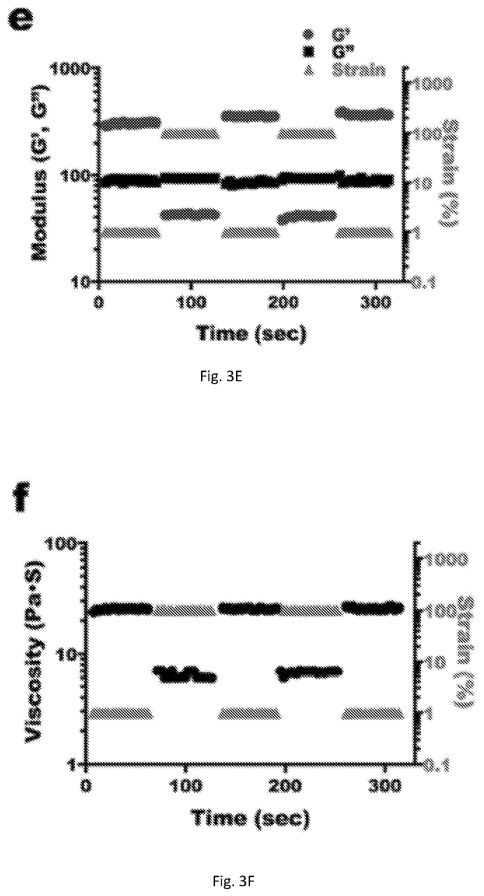

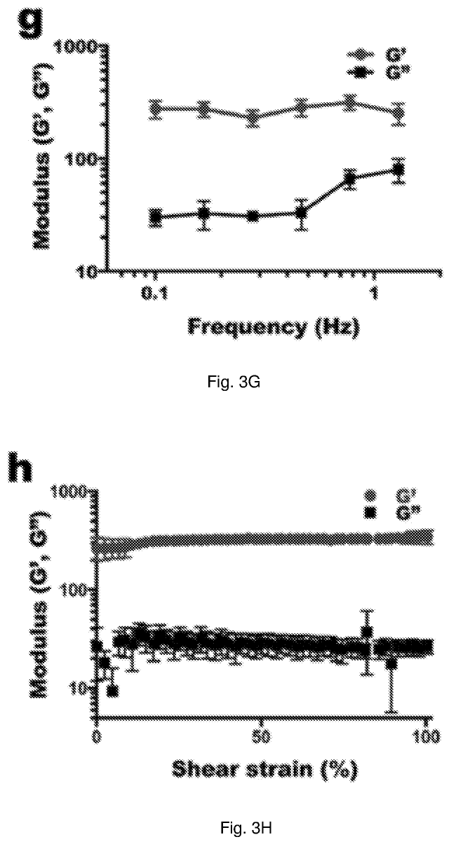

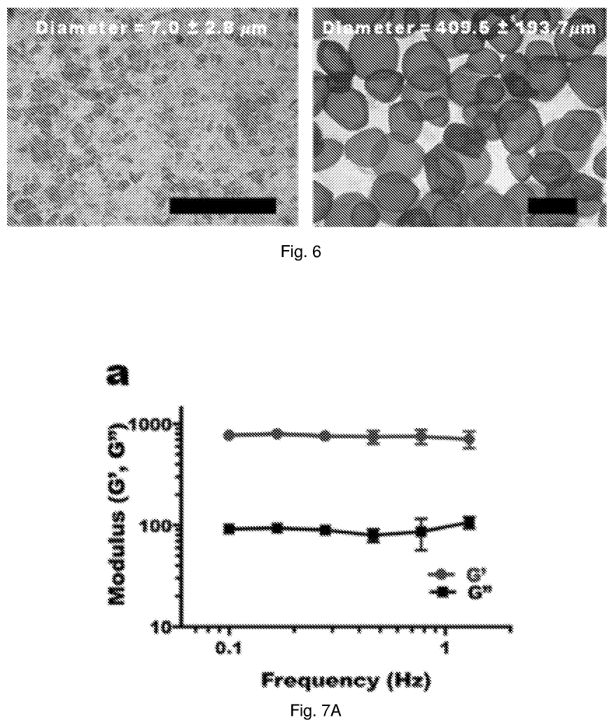

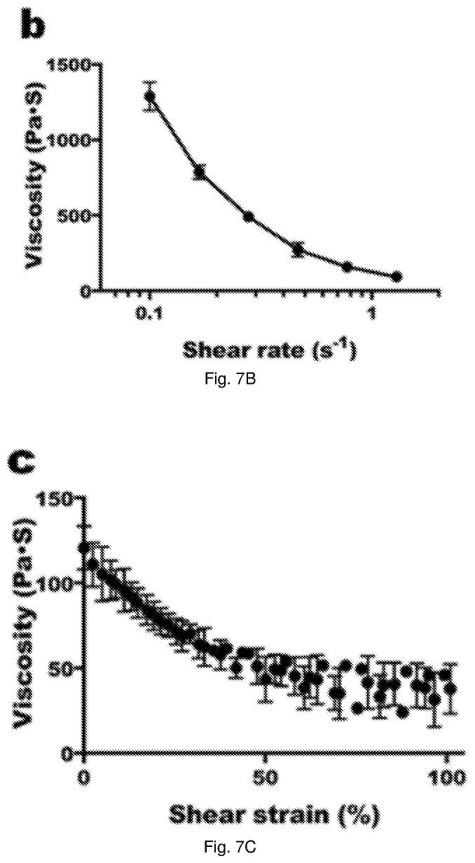

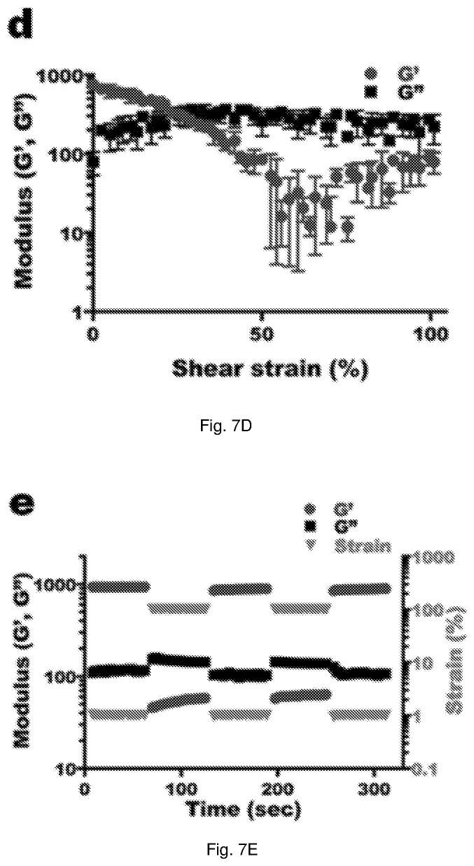

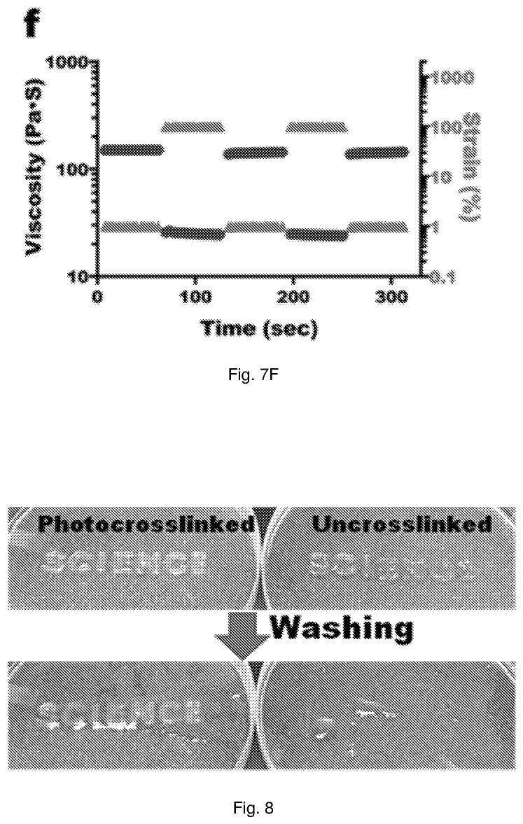

To identify favorable properties of alginate microgels for use as supporting medium for 3D cell printing, several rheological measurements were performed on supporting medium made up of two different sizes of alginate microgels (FIG. 3 and FIG. 7). To verify the solid-like properties of alginate microgel supporting medium, a frequency sweep at low strain amplitude (1%) was conducted, measuring the elastic and viscous shear moduli and viscosity. The data show both sizes (7.0±2.8 and 409.5±193.7 μm, FIG. 6) of alginate microgels behave like solid materials at low shear strain due to the steric stabilization of highly packed microgels (FIG. 3A and FIG. 7A), but they exhibit shear-thinning properties with decreased viscosity as shear rate increases (FIG. 3B and FIG. 7B). To further identify the shear-thinning and shear yielding properties of the alginate microgel supporting medium in response to shear strain, the shear moduli with a strain sweep at a constant frequency (1 Hz) was measured. Both sizes of OMA microgels exhibited shear-thinning (FIG. 3C and FIG. 7C) and shear-yielding (FIG. 3D and FIG. 7D) properties following increased shear strain application. Although both sizes of microgels exhibited a crossover at similar strain amplitude, the modulus at the crossover point (G′=G″) of the smaller OMA microgels was much lower than that of the larger OMA microgels (FIG. 3D and FIG. 7D in ESI†). To characterize the self-healing or recovery behavior of the alginate microgel medium, dynamic strain tests were performed with alternate low (1%) and high (100%) strains. A rapid recovery of the storage modulus (FIG. 2E and FIG. 6E) and viscosity (FIG. 2F and FIG. 6F) within seconds to the initial properties was repeatedly achieved over several cycles for both sizes of alginate microgels, indicating that the alginate microgel supporting medium can rapidly change from the solid to the fluid state via application of shear strain. Printing materials into viscoelastic supporting materials often results in crevasses created by the movement of the shaft of the dispensing needle and requires a third material that fills in crevasses. However, 3D structures of hMSCs can be written into alginate microgel supporting medium without creating crevasses due to the self-healing properties of the alginate microgel supporting medium. To evaluate the capacity of the OMA microgels to provide long-term support for 3D printed constructs, frequency (at 1% strain) and strain (at 1 Hz) sweep tests were conducted after photocrosslinking of the smaller sized OMA microgel-based supporting medium under low-level UV light. Frequency (FIG. 2G) and strain (FIG. 2H) sweeps exhibited significantly higher G′ than G″, indicating that photocrosslinked OMA microgel supporting medium is mechanically stable without shear yielding. The stability of photocrosslinked OMA microgel supporting medium was also confirmed by a wash out test (FIG. 7). While the photocrosslinked OMA microgel supporting medium remained stable on the Petri dish, uncrosslinked OMA microgel supporting medium could be easily removed by washing with water.

### Characterization of 3D Printed Cell-Only Filaments

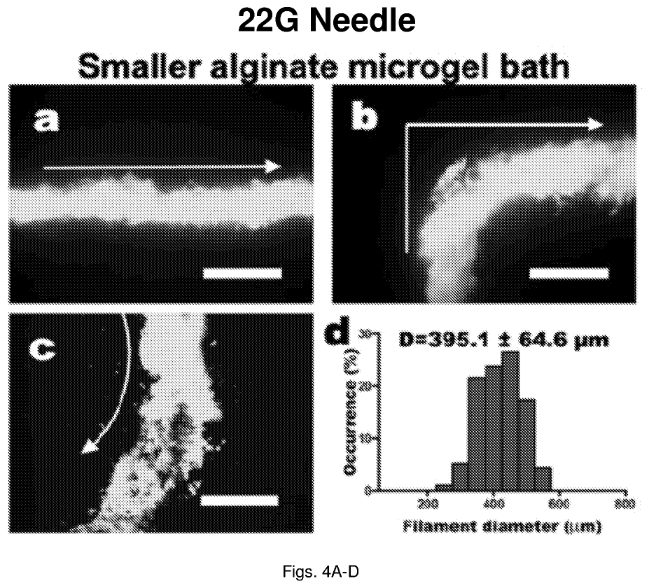

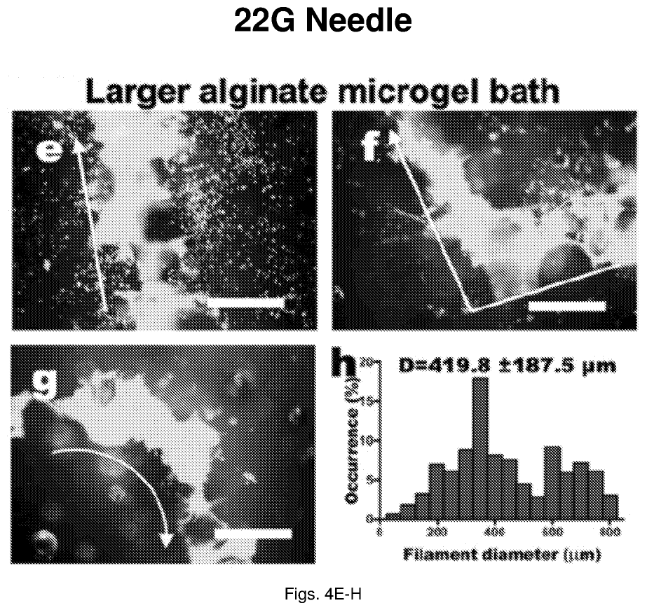

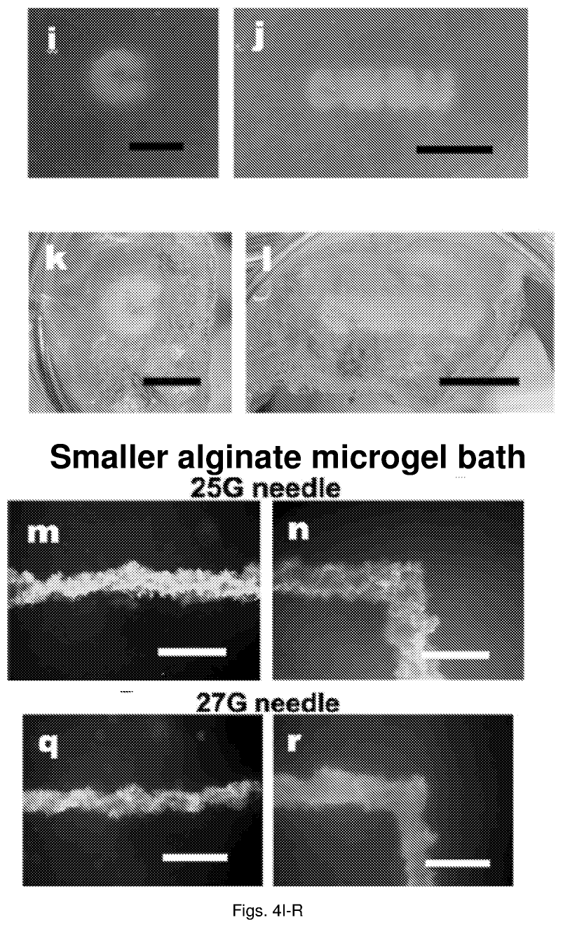

Next, it was important to determine the minimum printed structure feature size achievable using this strategy. Lines or “filaments” of cells were printed into supporting medium with both sizes of alginate microgels to compare resulting resolutions. Regardless of the microgel size, hMSCs in filaments exhibited high cell viability as visualized by live/dead assay, demonstrating no adverse effects of the bioprinting process and UV irradiation for curing the microgel supporting medium on cell survival (FIGS. 4A-C and E-F). The smaller alginate microgel supporting medium (FIG. 4D) exhibited higher resolution with narrow filament diameter distribution compared to the larger alginate microgel supporting medium (FIG. 4H), while the mean diameters of both hMSC filaments were similar (395.1±64.6 and 419.8±187.5 μm for filaments printed in small and larger microgel supporting medium, respectively). Since medium pores result from the space between the microgels, larger microgels make larger medium pores and vice versa. Due to the larger pores, many hMSCs printed into the larger alginate microgel supporting medium dispersed into the medium from the filaments, while hMSC filaments printed into the smaller alginate microgel supporting medium show a limited dispersion of cells. Therefore, 3D printed hMSC constructs in the smaller alginate microgel supporting medium (FIGS. 4I and J) exhibit higher resolution than those in the larger alginate microgel supporting medium (FIGS. 4K and L). These results indicate that the supporting medium comprised of smaller alginate microgels, which has lower stiffness, yield strength and viscosity, is more favorable for printing hMSCs with high resolution. Importantly, when cells were printed into the smaller alginate microgel supporting medium with smaller-gauge needles (25 and 27 G), significantly higher resolution of hMSC filaments (p<0.05, one-way ANOVA with Tukey's multiple comparison test using GraphPad Prism) was achieved (FIG. 3M-R) compared to that with the larger-gauge needle (FIGS. 4A-C).

### 3D Printing of Complex Structures and Formation of Engineered Tissues

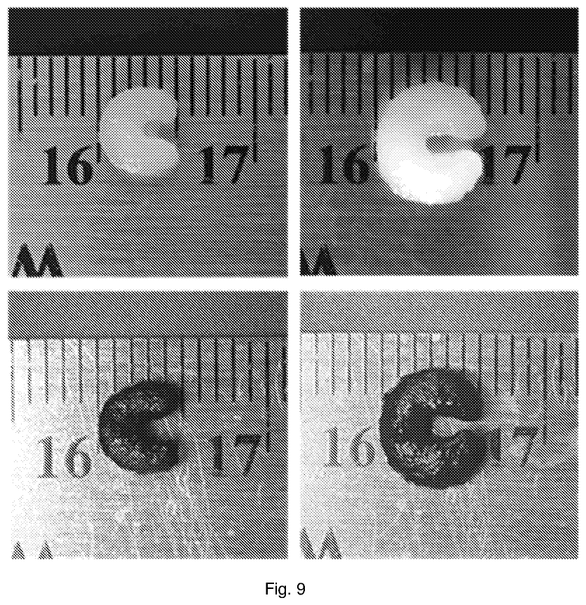

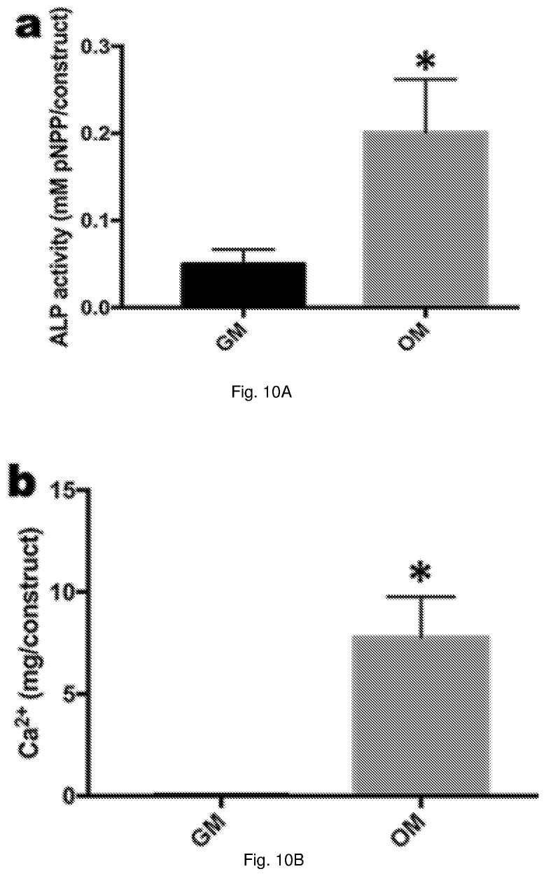

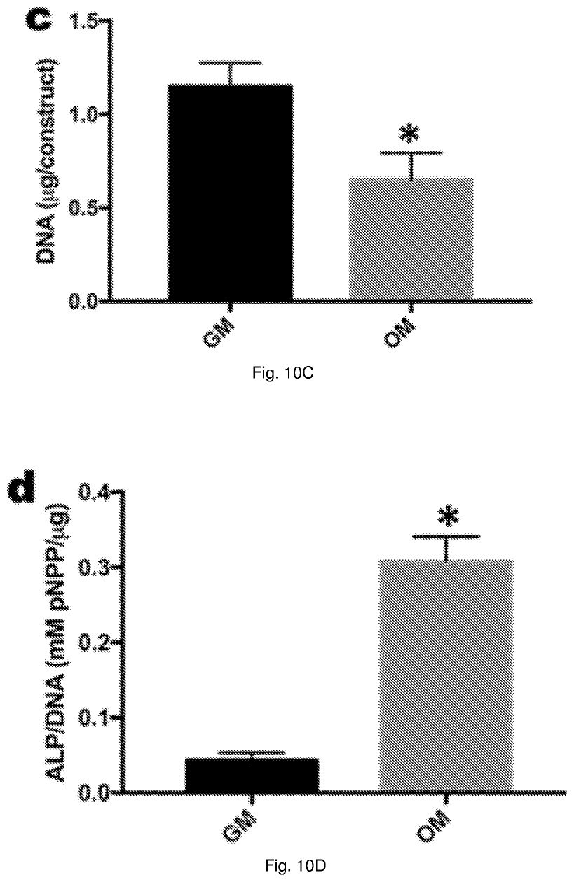

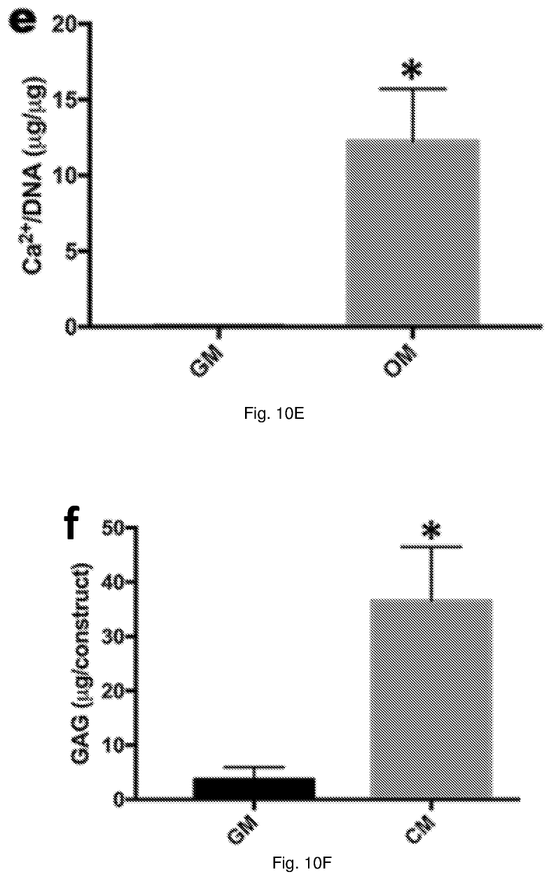

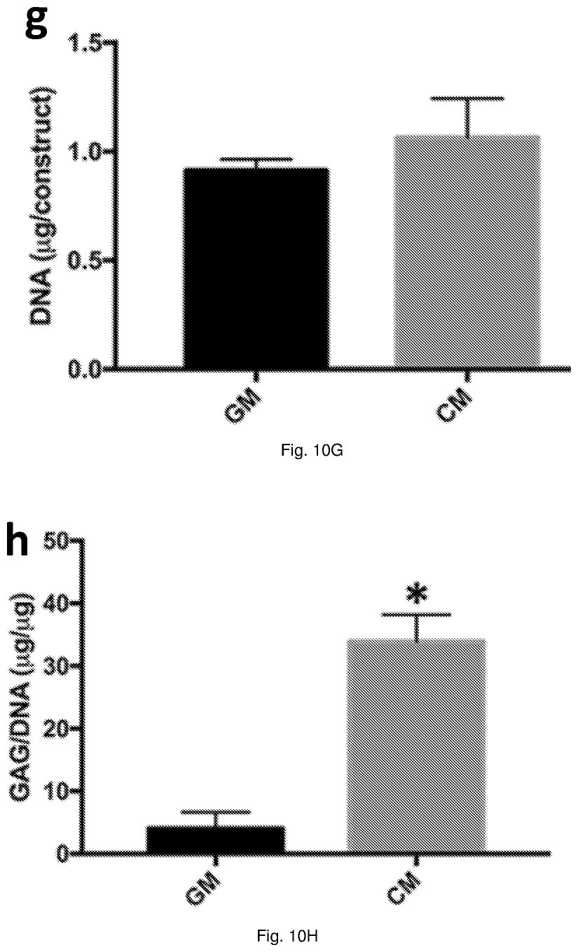

Long-term cell culture is essential to ensure tissue formation through maintenance cell-cell interactions, self-assembly into cellular condensations, and differentiation of the stem cells down desired lineages for engineering specific tissue types. Critical to achieving this for a cell-only bioink is the capacity to provide the mechanical stability with the supporting medium during the culture period. Since the alginate microgels possess photo-reactive methacrylate groups, the medium can be further photocrosslinked to form a more stable supporting structure that retains its shape for extended culture. After photocrosslinking, the alginate microgel supporting medium exhibited robust mechanical stability without shear yielding (FIG. 3H), maintained initial 3D printed structures (FIGS. 3C-F) and enabled long-term culture of 3D printed constructs for formation of functional tissue by differentiation of 3D bioprinted hMSCs. After 4 weeks of osteogenic or chondrogenic differentiation, formed tissue constructs were easily harvested from the alginate microgel supporting medium by applying shear force using a pipette. 3D printed hMSCs were assembled into precise multicellular structures following the architecture defined by computer-aided design (CAD) files (FIGS. 5A and D), and bone- (FIGS. 5B-C) or cartilage- (FIG. 4E-F and FIG. 8) like tissues were obtained in the photocrosslinked alginate microgel supporting medium. Differentiation down the osteogenic and chondrogenic lineages and resultant formation of bone and cartilage tissue were confirmed via Alizarin red (red) and Toluidine blue O (purple) staining, respectively; red and purple colors were intensively observed throughout the constructs (FIGS. 5C and F) and sectioned samples (FIGS. 5G and H). Lacunae structures were also observed in sectioned slides of chondrogenically differentiated constructs (FIG. 5H), indicating maturation of cartilage tissues. Successful tissue formation by the 3D printed hMSCs were further confirmed by quantification of osteogenic (i.e., alkaline phosphatase (ALP) activity and calcium deposition) and chondrogenic (i.e., glycosaminoglycan (GAG) production) markers (FIG. 9). Collectively, the microgel supporting medium allows not only high-resolution printing of cell-only bioink, but also provides printed construct mechanical stability after additional photocrosslinking, which permits culture of the constructs with stable structural maintenance and long-term differentiation in differentiation medium.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method for forming a scaffold-free 3D tissue construct comprising:
providing a self-healing, shear thinning, crosslinkable, biocompatible hydrogel support medium;
printing a first bioink into the hydrogel support medium, the first bioink including a plurality of cells and optional macromer carrier, nanoparticles, microparticles, bioactive agents, cell aggregates, and/or organoids, the printed first bioink having a defined shape; and
culturing the printed plurality of cells and optional macromer carrier, nanoparticles, microparticles, bioactive agents, cell aggregates, and/or organoids in the hydrogel support medium to form a cell aggregate or tissue construct with the defined shape.

2. The method of claim 1, wherein the hydrogel support medium maintains the defined shape of the printed first bioink during printing and optionally culturing.

3. The method of claim 1, wherein the hydrogel support medium behaves as a viscous fluid during printing and as is resistant to flow before and after printing.

4. The method of claim 1, further comprising crosslinking the hydrogel support medium printed with the first bioink to enhance the mechanical stability of the hydrogel support medium.

5. The method of claim 4, further comprising separating the printed construct from the hydrogel support medium.

6. The method of claim 1, wherein the hydrogel support medium comprises a plurality of hydrogel particles that include a plurality of crosslinkable biodegradable natural polymer macromers.

7. The method of claim 5, the hydrogel particles having an average diameter of about 10 nm to about 10 mm.

8. The method of claim 6, wherein the natural polymer macromers are at least partially crosslinked.

9. The method of claim 6, the natural polymer macromers include a plurality of acrylated and/or methacrylated natural polymer macromers.

10. The method of claim 9, wherein the acrylated and/or methacrylated, natural polymer macromers are polysaccharides, which are optionally oxidized to aldehyde saccharide units.

11. The method of claim 6, wherein the natural polymer macromers are ionically crosslinked.

12. The method of claim 6, wherein the natural polymer macromers are photocrosslinkable to enhance the mechanical stability of the hydrogel support medium.

13. The method of claim 6, the natural polymer macromers comprising oxidized, acrylated and/or methacrylated alginates.

14. The method of claim 1, wherein the hydrogel is cytocompatible and, upon degradation, produces substantially non-toxic products.

15. The method of claim 1, wherein the plurality of cells comprises progenitor cells, undifferentiated cells, differentiated cells, and/or cancer cells.

16. The method of claim 1, wherein the plurality of cells include mesenchymal stem cells.

17. The method of claim 1, wherein the first bioink is free of or substantially free of the optional macromer carrier, nanoparticles, microparticles, bioactive agents, cell aggregates, and/or organoids.

18. The method of claim 1, wherein the first bioink is in a liquid or slurry form during printing.

19. The method of claim 1, wherein the hydrogel support medium and printed bioink is provided in a culture medium.

20. The method of claim 19, wherein the culture medium comprises a cell differentiation medium.

21. The method of claim 1, further comprising printing a second bioink into the hydrogel support medium, wherein the second bioink is different than the first bioink and includes a plurality of cells, cell aggregates, a macromer carrier, nanoparticles, microparticles, bioactive agents, organoids, and/or combinations thereof.

22-35. (canceled)
